(19) United States
(12) Patent Application Publication
(10) Pub. No.: US 2025/0378937 A1
(43) Pub. Date: Dec. 11, 2025

(54) VIRTUAL TEST PLATFORM FOR PREDICTING A SPECIFIC COGNITIVE-AFFECTIVE STATE OF AN INDIVIDUAL

(71) Applicant: GM Global Technology Operations LLC, Detroit, MI (US)

(72) Inventors: Gina Notaro, Los Angeles, CA (US); Dalong Gao, Rochester, MI (US); Miguel A. Saez, Clarkston, MI (US); Ryan Mustari, Venice, CA (US); Arya Ketabchi Haghighat, Woodland Hills, CA (US); Vahidreza Molazadeh, Royal Oak, MI (US)

(21) Appl. No.: 18/738,588

(22) Filed: Jun. 10, 2024

(51) Int. Cl. G16H 20/70; G06F 3/01; G16H 10/20; G16H 50/20

(52) U.S. Cl. CPC G16H 20/70; G06F 3/011; G16H 10/20; G16H 50/20

(57) ABSTRACT

A virtual test platform for predicting a specific cognitive-affective state of an individual includes a simulated environment generator that creates a computer-generated environment representing a workspace viewed by the individual, where the individual is required to complete an assigned task within the workspace simulated by the computer-generated environment. The virtual test plate also includes at least one of the following: one or more physiological sensors that monitor physiological measurements of the individual and an input device receiving user input generated by the individual, where the individual answers one or more survey questions either while performing or after performing the assigned task by the input device. The virtual test platform includes one or more controllers in electronic communication with the simulated environment generator, the one or more physiological sensors, and the input device.

### INTRODUCTION

The present disclosure relates to a virtual test platform for predicting a specific cognitive-affective state of an individual while the individual completes an assigned task within a workspace created by a simulated environment generator.

Various augmented and virtual reality platforms currently exist and may be employed in a variety of applications. Augmented reality is an interactive experience that augments a live view of a real-world environment with computer generated perceptual information such as, for example, graphics, video, and sound. In contrast, virtual reality is an immersive experience that completely replaces a real-world environment with a simulated one.

Some types of existing augmented and virtual reality platforms are directed towards training and familiarizing an individual with an assigned task or technologies related to applications such as, but not limited to, gaming, sports, and medical treatment. However, existing augmented and virtual reality platforms do not determine the individual's cognitive-affective state while performing the assigned task. Instead, the current approach to determine an individual's cognitive-affective state while completing an assigned task involves fabricating a real-world physical test platform. It is to be appreciated that building or creating the physical test platform may be time-consuming, requires numerous resources, and often involves various modifications to test different types of use cases and different user profiles.

Thus, while current approaches to determine an individual's cognitive-affective state while completing an assigned task achieve their intended purpose, there is a need in the art for an improved approach for determining an individual's cognitive-affective state while completing the assigned task.

### SUMMARY

According to several aspects, a virtual test platform for predicting a specific cognitive-affective state of an individual is disclosed. The virtual test platform includes a simulated environment generator that creates a computer-generated environment representing a workspace viewed by the individual, where the individual is required to complete an assigned task within the workspace simulated by the computer-generated environment. The virtual test platform also includes at least one of the following: one or more physiological sensors that monitor physiological measurements of the individual and an input device receiving user input generated by the individual, where the individual answers one or more survey questions either while performing or after performing the assigned task by the input device. The virtual test platform also includes one or more controllers in electronic communication with the simulated environment generator, the one or more physiological sensors, and the input device. The one or more controllers include one or more processors that execute instructions to instruct the simulated environment generator to create the computer-generated environment representing the workspace. The one or more controllers predict the specific cognitive-affective state of the individual as the individual completes the assigned task created by the simulated environment generator based on at least one of the following: the physiological measurements from the one or more physiological sensors and user input received from the input device indicative of answers to the one or more survey questions from the individual. The one or more controllers formulate one or more recommendations to implement the workspace in a real-world environment based on the specific cognitive-affective state of the individual.

In another aspect, the one or more processors of the one or more controllers execute instructions to instruct the simulated environment generator to modify one or more experimental parameters related to the assigned task within the workspace, where the individual is required to re-execute the assigned task within the workspace when the one or more experimental parameters that are related to the assigned task are modified.

In yet another aspect, the specific cognitive-affective state of the individual is predicted based on the one or more survey questions.

In an aspect, the one or more processors of the one or more controllers execute instructions to instruct the simulated environment generator to create computer-generated perception information that asks the individual the one or more survey questions each time the one or more experimental parameters within the workspace are modified.

In another aspect, the one or more survey questions include a plurality of multiple-choice answers, and wherein each multiple-choice answer includes a numerical level representing a magnitude of the specific cognitive-affective state experienced by the individual.

In yet another aspect, the one or more survey questions include free-text responses.

In an aspect, the specific cognitive-affective state of the individual is predicted based on the physiological measurements collected by the one or more physiological sensors.

In another aspect, the one or more processors of the one or more controllers execute instructions to continue to monitor the physiological measurements of the individual collected by the one or more physiological sensors as the one or more experimental parameters are modified.

In yet another aspect, the one or more physiological sensors include one or more of the following: off-body physiological sensors and on-body physiological sensors.

In an aspect, the off-body physiological sensors include one or more of the following: audio sensors, cameras, thermal cameras, body markers, facial markers, pressure mats, and technology tracking sensors.

In another aspect, the on-body physiological sensors include one or more of the following: pressure sensors, galvanic skin response sensors, eye-tracking sensors, electroencephalography (EEG) sensors, electromyography (EMG) sensors, and functional near-infrared spectroscopy (FNIRS) sensors.

In yet another aspect, the one or more processors of the one or more controllers execute instructions to classify the physiological measurements of the individual into categories indicating the specific cognitive-affective state of the individual based on signal values generated by the one or more physiological sensors.

In an aspect, the one or more processors of the one or more controllers execute instructions to assign numerical values to signals generated by the one or more physiological sensors, wherein the numerical values represent the magnitude of the specific cognitive-affective state and index numerical values to a baseline value that represents a neutral cognitive-affective state, where the specific cognitive-affective state predicted by the one or more controllers is relative to the neutral specific cognitive-affective state.

In another aspect, the specific cognitive-affective state of the individual is predicted based on both the physiological measurements collected by the one or more physiological sensors and the one or more survey questions.

In yet another aspect, the one or more processors of the one or more controllers execute instructions to predict the specific cognitive-affective state of the individual based on a custom classification model is based on a recurrent neural network (RNN) having an internal memory.

In an aspect, the one or more recommendations include ranges of values for the one or more experimental parameters that result in the individual having a neutral cognitive-affective state while completing the assigned task within the workspace.

In another aspect, a virtual test platform for predicting a specific cognitive-affective state of an individual. The virtual test platform includes a simulated environment generator that creates a computer-generated environment representing a workspace viewed by the individual, where the individual is required to complete an assigned task within the workspace simulated by the computer-generated environment. The virtual test platform also includes an input device receiving user input generated by the individual, where the individual answers one or more survey questions either while performing or after performing the assigned task by the input device. The virtual test platform also includes one or more controllers in electronic communication with the simulated environment generator and the input device, where the one or more controllers include one or more processors that execute instructions to instruct the simulated environment generator to create the computer-generated environment representing the workspace. The one or more controllers predict the specific cognitive-affective state of the individual as the individual completes the assigned task created by the simulated environment generator based on user input received from the input device indicative of answers to the one or more survey questions from the individual. The one or more controllers instruct the simulated environment generator to modify one or more experimental parameters related to the assigned task within the workspace, where the individual is required to re-execute the assigned task within the workspace when the one or more experimental parameters that are related to the assigned task are modified. The one or more controllers instruct the simulated environment generator to create computer-generated perception information that asks the individual the one or more survey questions each time the one or more experimental parameters within the workspace are modified. The one or more controllers formulate one or more recommendations to implement the workspace in a real-world environment based on the specific cognitive-affective state of the individual.

In another aspect, the one or more survey questions include a plurality of multiple-choice answers, and where each multiple-choice answer includes a numerical level representing a magnitude of the specific cognitive-affective state experienced by the individual.

In yet another aspect, the one or more survey questions include free-text responses.

In an aspect, a virtual test platform for predicting a specific cognitive-affective state of an individual is disclosed. The virtual test platform includes a simulated environment generator that creates a computer-generated environment representing a workspace viewed by the individual, where the individual is required to complete an assigned task within the workspace simulated by the computer-generated environment. The virtual test platform also includes one or more physiological sensors that monitor physiological measurements of the individual and one or more controllers in electronic communication with the simulated environment generator and the one or more physiological sensors. The one or more controllers include one or more processors that execute instructions to instruct the simulated environment generator to create the computer-generated environment representing the workspace. The one or more controllers predict the specific cognitive-affective state of the individual as the individual completes the assigned task created by the simulated environment generator based on the physiological measurements from the one or more physiological sensors. The one or more controllers instruct the simulated environment generator to modify one or more experimental parameters related to the assigned task within the workspace, where the individual is required to re-execute the assigned task within the workspace when the one or more experimental parameters that are related to the assigned task are modified. The one or more controllers continue to monitor the physiological measurements of the individual collected by the one or more physiological sensors as the one or more experimental parameters are modified. The one or more controllers formulate one or more recommendations to implement the workspace in a real-world environment based on the specific cognitive-affective state of the individual.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a schematic diagram of the disclosed virtual test platform including one or more controllers in electronic communication with a simulated environment generator, according to an exemplary embodiment; and

FIG. 2 is a schematic diagram of an exemplary workspace that is created by the simulated environment generator shown in FIG. 1, according to an exemplary embodiment.

### DETAILED DESCRIPTION

The following description is merely exemplary in nature and is not intended to limit the present disclosure, application, or uses.

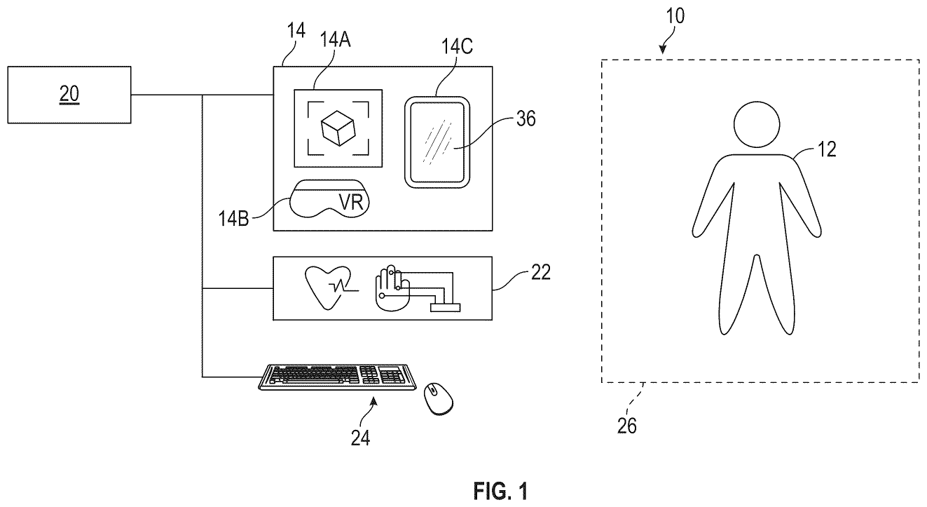

Referring to FIG. 1, an exemplary virtual test platform 10 for predicting a specific cognitive-affective state of an individual 12 while completing an assigned task created by a simulated environment generator 14 is illustrated. The virtual test platform 10 includes one or more controllers 20 in electronic communication with the simulated environment generator 14. In addition to the simulated environment generator 14, the one or more controllers 20 are also in electronic communication with at least one of the following: one or more physiological sensors 22 and an input device 24. The simulated environment generator 14 creates a computer-generated environment that represents a workspace 26 viewed by the individual 12, where the individual 12 is required to complete the assigned task within the workspace 26 created by the simulated environment generator 14.

As explained below, the disclosed virtual test platform 10 predicts the specific cognitive-affective state of the individual 12 as the individual 12 completes the assigned task within the workspace 26 created by the simulated environment generator 14. The virtual test platform 10 may also formulate one or more recommendations to implement the workspace 26 in a real-world environment based on the specific cognitive-affective state of the individual 12 while completing the assigned task. As explained below, the specific cognitive-affective state of the individual 12 is predicted based on physiological measurements collected by the one or more physiological sensors 22, survey data completed by the individual 12, or both the physiological measurements and the survey data. Some examples of the specific cognitive-affective state predicted by the virtual test platform 10 include, but are not limited to, comfort, fatigue, cognitive workload, and stress.

The simulated environment generator 14 may be any device that creates the computer-generated environment the represents the workspace 26 such as, but not limited to, an augmented reality system 14A, a virtual reality system 14B, or a computing device 14C including a display 36. The augmented reality system 14A may include, for example, a computing device that includes a display or a pair of smart glasses that overlays computer-generated perception information such as images and text onto the real-world environment to create the computer-generated environment. The virtual reality system 14B may include, for example, a smart headset, a smart helmet, or a display that is part of a computing device that shows a completely virtual computer-generated environment representing the workspace 26. The computing device 14C may be any type of computing device such as a laptop or tablet computer, where the computer-generated environment is shown upon the display 36. The individual 12 views the computer-generated environment created by the simulated environment generator 14 and completes the assigned task within the workspace 26.

The workspace 26 is a simulated or computer-generated environment representing an area where the individual 12 is required to complete the assigned task such as, but not limited to, a manufacturing environment, a flight simulation environment for controlling an aircraft, or a driving simulation environment for controlling a vehicle. Some examples of the assigned task include, for example, flying the aircraft by manipulating flight controls such as a joystick or yoke control, and driving a vehicle by manipulating driver inputs such as the steering wheel, brake pedal, and accelerator pedal. Some examples of manufacturing environments that may be simulated include, but are not limited to, a robot-assisted door assembly line, a robot-assisted jack assembly line, a robot-assisted sub-component assembly line, a robot-assisted battery assembly line, a robot-assisted end-of-line assembly, and a robot-assisted engine assembly line.

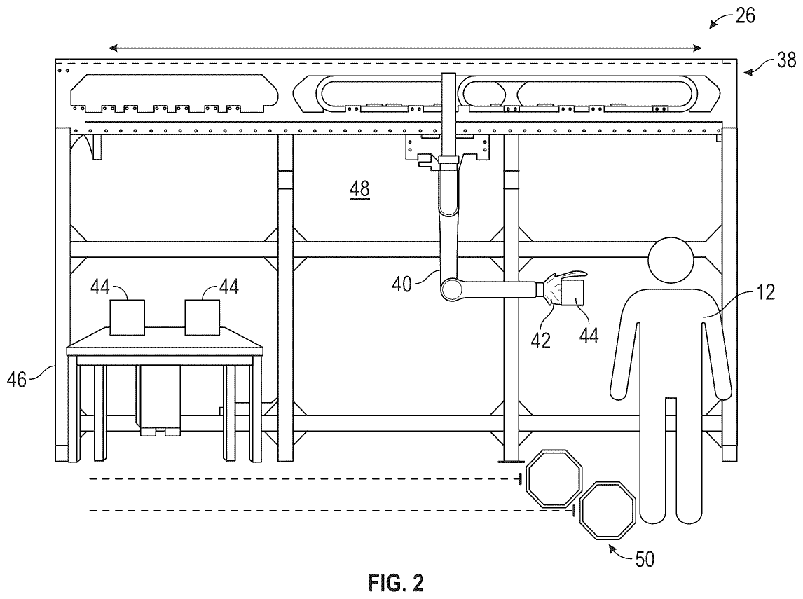

In the example as shown in FIG. 2, the workspace 26 includes a robotic arm 40 including a gripper 42, where the robotic arm 40 is guided by an overhead rail system 38. The robotic arm 40 retrieves a colored block 44 from a table 46 located on an opposite side of a room 48 from where the individual 12 is positioned, travels across the room 48 by the overheard rail system 38 to the individual 12, hands the colored block 44 to the individual 12, and retrieves another colored block 44 from the table 46. In the example as illustrated, the assigned task involves having the individual 12 receive the colored block 44 from the robotic arm 40, memorize a sequence of colored blocks 44 handed to him or her by the robotic arm 40, and recite the sequence of colored blocks 44 aloud.

Referring to FIG. 1, the one or more physiological sensors 22 include any type of sensor for monitoring physiological measurements that indicate the specific cognitive-affective state of the individual 12, and may include off-body physiological sensors, on-body physiological sensors, or both off-body physiological sensors and on-body physiological sensors. Some examples of off-body physiological sensors include, but are not limited to, audio sensors such as a microphone for detecting speech and sound, cameras that monitor the scene, body pose, and movement of the individual 12, thermal cameras for detecting the heat profile of the individual 12, body markers that detect body pose and movement of the individual 12, facial markers that detect facial expressions of the individual 12, pressure mats that detect weight shifting, and technology tracking sensors that monitor the use, behavior, and interactions between the individual 12 and a specific device being employed by the individual 12. The body markers and the facial markers are tracked by cameras and are processed by algorithms to determine the body pose and facial expressions of the individual. The technology tracking sensors may include any type of device that receives user-generated input from the individual 12 such as, but not limited to, a touchscreen, a virtual reality controller, a keyboard, a computer mouse, or a stylus.

Some examples of on-body physiological sensors include, but are not limited to, pressure sensors for measuring heart rate and respiration, galvanic skin response sensors for detecting emotional arousal, eye-tracking sensors that detect gaze and gaze direction, electroencephalography (EEG) sensors for detection electrical activity from the brain, electromyography (EMG) sensors for detecting muscle activity of the individual, and functional near-infrared spectroscopy (FNIRS) sensors that measure physiological data such as respiration and the concentration of oxy-hemoglobin (O2Hb) and deoxy-hemoglobin (Hhb) in the prefrontal cortex of the individual 12.

It is to be appreciated that the specific physiological sensors 22 included by the virtual test platform 10 depend upon the specific cognitive-affective state being evaluated. This is because different types of cognitive-affective states elicit unique and distinct physiological responses from the individual 12. For example, specific cognitive-affective states such as excitement and fear result in physiological effects such as high heart rate, and therefore pressure sensors for monitoring heart rate may be included. In the example as illustrated in FIG. 2, the specific cognitive-affective state of the individual 12 being evaluated by the virtual test platform 10 is comfort. Accordingly, the one or more physiological sensors 22 include a camera, a microphone, a galvanic skin response sensor, and pressure sensors for monitoring heart rate.

Referring to FIG. 1, the input device 24 represents any device for receiving user input generated by the individual 12 such as, for example, a touchscreen, a keyboard, a computer mouse, or an audio receiver that captures sounds and words spoken by the individual 12. In one embodiment, the input device 24 represents a technology tracking sensor that is part of the off-body physiological sensors. In one embodiment, the one or more controllers 20 instruct the simulated environment generator 14 to create computer-generated perception information that asks one or more survey questions to the individual 12. The one or more controllers 20 receive user input from the input device 24 indicative of the answers to the one or more survey questions from the individual 12. For example, the simulated environment generator 14 may display computer-generated text upon a display that is part of the simulated environment generator 14 asking the individual 12 the one or more survey questions. In another example, the simulated environment generator 14 may generate an audible voice over a speaker asking the individual 12 the one or more survey questions.

The individual 12 answers one or more survey questions either while or after performing the assigned task by entering his or her answer by the input device 24. In an embodiment, instead of entering the answers to the one or more survey questions into the input device 24, the individual 12 may answer the one or more survey questions manually by completing paperwork, and the user input is then entered by a third party employing the input device 24. In another implementation, the user input may be entered using approaches other than a third party such as, for example, scanning a document for information based on optical mark recognition (OMR) or optical character recognition (OCR).

The one or more survey questions relate to and are probative of the specific cognitive-affective state of the individual 12. In one embodiment, the one or more survey questions include a plurality of multiple-choice answers, where each multiple-choice answer includes a numerical level representing the magnitude of the specific cognitive-affective state experienced by the individual 12. Some examples of predefined numerical scales representing the specific cognitive-affective state of an individual include, but are not limited to, the National Aeronautics and Space Administration Task Load Index (TLX), the instantaneous self-assessment (ISA), and the multidimensional fatigue inventory (MFI). Alternatively, in another embodiment, the one or more survey questions include free-text responses. The one or more controllers 20 may execute one or more natural language processing (NLP) algorithms to extract word content, semantics, and valence to determine the specific cognitive-affective state of the individual 12 while completing the assigned task based on the free-text response.

It is to be appreciated that the one or more controllers 20 instruct the simulated environment generator 14 to modify one or more experimental parameters related to the assigned task within the workspace 26, where the individual 12 is required to re-execute the assigned task within the workspace 26 with the one or more experimental parameters that are related to the assigned task modified. The one or more experimental parameters modify the workspace 26 where the individual 12 completes the assigned task. Each experimental parameter has the potential to impact the specific cognitive-affective state of the individual 12 as the individual 12 completes the assigned task.

In the example as shown in FIG. 2, the one or more experimental parameters include the speed that the robotic arm 40 travels while being guided by the overhead rail system 38 and a stopping distance 50 measured between the robotic arm 40 and the individual 12, and the specific cognitive-affective state of the individual 12 is comfort. As the speed of the robotic arm 40 is increased and as the stopping distance 50 between the robotic arm 40 and the individual 12 is decreased, the comfort level of the individual 12 may decrease.

The one or more controllers 20 instruct the simulated environment generator 14 to create computer-generated perception information asking the individual 12 the one or more survey questions each time the one or more experimental parameters within the workspace 26 are modified. Similarly, the one or more controllers 20 continue to monitor the physiological measurements of the individual 12 collected by the one or more physiological sensors 22 as the one or more experimental parameters are modified. In the example as shown in FIG. 2, it is to be appreciated that since the specific cognitive-affective state is comfort, the one or more survey questions include the query of “please rate your overall comfort (or discomfort) level with working with your robot teammate in the scenario you just experienced,” and includes nine numerically scaled answers ranging from −4 to 4, where −4 indicates extreme discomfort and 4 indicates extreme comfort. It is to be appreciated that the numerically scaled answers allow for the one or more controllers 20 to determine a relative change in comfort while changing the one or more experimental parameters within the workspace 26. Additionally, in an embodiment, one of the survey questions includes the query of “would you work with the robotic arm in a real-world setting?”.

The one or more controllers 20 predict the specific cognitive-affective state of the individual 12 based on either the physiological measurements of the individual 12 monitored by the one or more physiological sensors 22, the one or more survey questions created by the simulated environment generator 14, or both the physiological measurements and the survey questions. In an embodiment where only the one or more survey questions are considered when predicting the specific cognitive-affective state of the individual 12, the one or more survey questions each include the multiple-choice answers, and where each answer of the plurality of multiple-choice answers includes a numerical level representing the magnitude of the specific cognitive-affective state experienced by the individual 12, the one or more controllers 20 employ one or more statistical approaches to predict the specific cognitive-affective state of the individual 12. Specifically, the one or more controllers 20 employ one or more statistical approaches to determine a change in the magnitude of the specific cognitive-affective state of the individual 12 reflected in the plurality of multiple-choice answers provided by the individual 12 while the one or more experimental parameters related to the assigned task within the workspace 26 are modified. It is to be appreciated that responses to the multiple-choice answers may be reduced to a single metric for each experimental parameter based on statistical approaches such as, for example, a t-test comparison of continuous ratings from two or more different experimental parameters.

In an embodiment where only the physiological measurements of the individual 12 monitored by the one or more physiological sensors 22 is considered when predicting the specific cognitive-affective state of the individual 12, the one or more controllers 20 predict the specific cognitive-affective state of the individual 12 based on either a classification model or a thresholding technique that considers numerical values representing the magnitude of the specific cognitive-affective state of the individual 12. Specifically, the classification model is any type of supervised machine learning technique that classifies the physiological measurements of the individual 12 into categories indicating the specific cognitive-affective state of the individual 12 based on signals generated by the one or more physiological sensors 22. Some examples of classification models that may be used include, but are not limited to, decision tree classifiers, multinomial logistic regression models, and support vector machine (SVM) classification.

Alternatively, the one or more controllers 20 predict the specific cognitive-affective state of the individual 12 based on the numerical values that represent the magnitude of the specific cognitive-affective state of the individual 12. Specifically, the one or more controllers 20 may assign the numerical values to signals generated by the one or more physiological sensors 22. The numerical values assigned to the signals that represent the magnitude of the specific cognitive-affective state are indexed to a baseline value representing a neutral cognitive-affective state, and the specific cognitive-affective state predicted by the one or more controllers 20 is relative to the neutral specific cognitive-affective state. For example, if the numerical value assigned to the signals generated by one of the physiological sensors 22 includes a value of 5.7, the baseline value is 2, and the specific cognitive-affective state is stress, then the one or more controllers 20 may predict the specific cognitive-affective state of the individual 12 as an elevated level of stress.

The one or more controllers 20 may predict the specific cognitive-affective state of the individual 12 based on physiological measurements collected at a single point in time or, in the alternative, collected over a period of time. It is also to be appreciated that the one or more controllers 20 may predict the specific mental state of the individual 12 based on regularly sampled data streams collected from the one or more physiological sensors 22, irregularly sampled data streams collected from the one or more physiological sensors 22, response times of the individual 12, a length of time the individual 12 engages with a device, or task-related outcomes. An example of a regularly sampled data stream would include output from optical sensors that measure heart rate, while an irregularly sampled data stream would include a movement tracker that only detects changes in movement of the individual 12, or an object that the individual 12 intermittently manipulates, such as a computer mouse.

The one or more controllers 20 may execute one or more preprocessing algorithms to remove extraneous information from the physiological measurements collected by the one or more physiological sensors 22. Some examples of the preprocessing algorithms include, but are not limited to, outlier removal, time slicing to include only relevant time periods, resampling the data streams to the same frequency, removal of data artifacts, signal bias and offset removal, and removal of extraneous frequencies.

In one embodiment where the physiological data is collected at a single point in time, the one or more controllers 20 may employ one or more standard statistical approaches such as mean and standard deviation to determine differences in the physiological data between different subsets of experimental parameters or other conditions such as time windows when the stimuli occurred. In an embodiment where the physiological data is collected over a period of time and includes timestamps, the one or more controllers 20 may aggregate the physiological data over a binned window of time, where the binned windows are directly input into a classification model such as a decision tree, logistic regression, or SVM for determining the presence of absence of a specific cognitive-affective state, and computing the specific cognitive-affective state as a time series. It is to be appreciated that the binned windows may include aggregated window values including overlapping and non-overlapping windows. This approach may be used to determine the percentage of time during a recording session that the individual was in the specific cognitive-affective state. In an embodiment where the physiological data includes a waveform that fluctuates over time, such as physiological data collected by EEG sensors or EMG sensors, the one or more controllers 20 employ one or more modeling approaches that consider the temporal dependency of past data points such as, but not limited to, a long short-term memory (LSTM) model, other types of hidden recurrent neural networks (RNNs), and hidden Markov models. In an embodiment where the physiological data includes image data that represents the pose of the individual 12, the one or more controllers 20 may include one or more convolutional neural networks (CNNs) to determine the pose of the individual 12.

In embodiments, the one or more controllers 20 may employ one or more signal processing techniques to extract features from the physiological measurements for determining the specific cognitive-affective state such as, but not limited to, time-series analysis and frequency analysis. Some examples of time-series analysis include binned signal magnitude, peak detection, signal bias determination, and standard deviation, and some examples of frequency analysis include Fourier analysis, frequency band filtering, power analysis, and frequency ratios. As an example, if the physiological measurements include EEG data, then the physiological signals of interest include time-locked event-related potentials (ERPs), which are peaks in the data that vary in amplitude and timing depending on the specific cognitive-affective state. Frequency information can be derived from the EEG data by considering power levels and/or ratios of well-characterized frequency bands, all with different cognitive and emotional state implications depending on which spatial regions of the individual's scalp the EEG signals originate from. The well-characterized frequency bands commonly include delta (0.5-4 Hertz), theta (4-8 Hertz), alpha (8-13 Hertz), beta (13-30 Hertz), and gamma (>30 Hertz) waveforms.

In an embodiment where both the physiological measurements of the individual 12 monitored by the one or more physiological sensors 22 and the one or more survey questions are considered when predicting the specific cognitive-affective state of the individual 12, the one or more controllers 20 may predict the specific cognitive-affective state of the individual 12 based on the approach described when only survey questions are considered, when only physiological data is considered, or based on a custom classification model. The custom classification model is based on any type of recurrent neural network (RNN) having an internal memory such as, for example, a LSTM model, a gated recurrent unit (GRU), a dual-attention time-aware GRU (DATA-GRU), a velocity-aware GRU (GRU-TV), an autoregressive moving average model (ARMA), and a generalized autoregressive conditional heteroskedasticity ARMA (ARMA-GARCH). Specifically, for example, in an embodiment where the physiological data collected by the one or more physiological sensors 22 is time-series data, the custom classification model is selected to consider the temporal order of the physiological data such as a LSTM model. It is to be appreciated that the custom classification model is customized for the specific workspace 26 and the specific cognitive-affective state that the virtual test platform 10 predicts.

In the event the custom classification model is created, the answers to the one or more survey questions may be used as ground truth data to train the custom classification model, where the physiological measurements collected by the one or more physiological sensors 22 are compared with respect to the ground truth data. In the present example, once the custom classification model is trained based on the ground truth data, the custom classification model may predict a numerical value representing the magnitude of the specific cognitive-affective state experienced by the individual 12 based on the physiological measurements collected by the one or more physiological sensors 22, or a classification of the specific cognitive-affective state experienced by the individual 12 based on subsequent physiological measurements collected by the one or more physiological sensors 22.

In one embodiment, the one or more controllers 20 compute a score for each prediction of the specific cognitive-affective state generated by the custom classification model that indicates either the accuracy of the numerical value representing the magnitude of the specific cognitive-affective state or the classification of the specific cognitive-affective state. The one or more controllers 20 may then build a confusion matrix based on the scores for each prediction of the specific cognitive-affective state predicted by the custom classification model. The confusion matrix indicates the accuracy or effectiveness of the specific cognitive-affective state predicted by the custom classification model. In one embodiment, if the confusion matrix indicates a satisfactory level of accuracy or effectiveness of the custom classification model, subsequent testing may only require monitoring the physiological sensors 22, without asking the individual 12 the one or more survey questions.

Once the virtual test platform 10 predicts the specific cognitive-affective state, the one or more controllers 20 then formulate one or more recommendations to implement the workspace 26 in a real-world environment based on the specific cognitive-affective state of the individual 12 while completing the assigned task. Specifically, the one or more recommendations include ranges of values for the one or more experimental parameters that result in the individual 12 having a neutral cognitive-affective state while completing the assigned task within the workspace 26. That is, in other words, the one or more recommendations suggest a range of values for the one or more experimental parameters that result in the individual 12 being comfortable or in a positive cognitive-affective state (e.g., low levels of stress or fatigue) while completing the assigned task within the workspace 26.

In the example as shown in FIG. 2, the one or more recommendations would include speeds for the robotic arm 40 and measurements related to the stopping distance 50 measured between the robotic arm 40 and the individual 12 that result in a neutral or enhanced level of comfort for the individual 12. As another example, if the workspace 26 represents an autonomous vehicle, then the one or more recommendations include values for experimental parameters such as turning speed, acceleration, braking, and merge distances that result in a neutral level of stress for the individual 12.

Referring generally to the figures, the disclosed virtual test platform for predicting the specific cognitive-affective state of an individual provides various technical effects and benefits. Specifically, the disclosed virtual test platform provides an approach to predict the specific cognitive-affective state of an individual completing an assigned task based on a virtual or augmented environment, without the need to invest in physical resources for building a physical workstation. It is also to be appreciated that the specific cognitive-affective state predicted by the virtual test platform may be used when developing a physical version of the workspace.

The controllers may refer to, or be part of an electronic circuit, a combinational logic circuit, a field programmable gate array (FPGA), a processor (shared, dedicated, or group) that executes code, or a combination of some or all of the above, such as in a system-on-chip. Additionally, the controllers may be microprocessor-based such as a computer having at least one processor, memory (RAM and/or ROM), and associated input and output buses. The processor may operate under the control of an operating system that resides in memory. The operating system may manage computer resources so that computer program code embodied as one or more computer software applications, such as an application residing in memory, may have instructions executed by the processor. In an alternative embodiment, the processor may execute the application directly, in which case the operating system may be omitted.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A virtual test platform for predicting a specific cognitive-affective state of an individual, the virtual test platform comprising:
a simulated environment generator that creates a computer-generated environment representing a workspace viewed by the individual, wherein the individual is required to complete an assigned task within the workspace simulated by the computer-generated environment;
at least one of the following: one or more physiological sensors that monitor physiological measurements of the individual and an input device receiving user input generated by the individual, wherein the individual answers one or more survey questions either while performing or after performing the assigned task by the input device; and
one or more controllers in electronic communication with the simulated environment generator, the one or more physiological sensors, and the input device, wherein the one or more controllers include one or more processors that execute instructions to:
instruct the simulated environment generator to create the computer-generated environment representing the workspace;
predict the specific cognitive-affective state of the individual as the individual completes the assigned task created by the simulated environment generator based on at least one of the following: the physiological measurements from the one or more physiological sensors and user input received from the input device indicative of answers to the one or more survey questions from the individual; and
formulate one or more recommendations to implement the workspace in a real-world environment based on the specific cognitive-affective state of the individual.

2. The virtual test platform of claim 1, wherein the one or more processors of the one or more controllers execute instructions to:
instruct the simulated environment generator to modify one or more experimental parameters related to the assigned task within the workspace, wherein the individual is required to re-execute the assigned task within the workspace when the one or more experimental parameters that are related to the assigned task are modified.

3. The virtual test platform of claim 2, wherein the specific cognitive-affective state of the individual is predicted based on the one or more survey questions.

4. The virtual test platform of claim 3, wherein the one or more processors of the one or more controllers execute instructions to:
instruct the simulated environment generator to create computer-generated perception information that asks the individual the one or more survey questions each time the one or more experimental parameters within the workspace are modified.

5. The virtual test platform of claim 3, wherein the one or more survey questions include a plurality of multiple-choice answers, and wherein each multiple-choice answer includes a numerical level representing a magnitude of the specific cognitive-affective state experienced by the individual.

6. The virtual test platform of claim 3, wherein the one or more survey questions include free-text responses.

7. The virtual test platform of claim 2, wherein the specific cognitive-affective state of the individual is predicted based on the physiological measurements collected by the one or more physiological sensors.

8. The virtual test platform of claim 7, wherein the one or more processors of the one or more controllers execute instructions to:
continue to monitor the physiological measurements of the individual collected by the one or more physiological sensors as the one or more experimental parameters are modified.

9. The virtual test platform of claim 7, wherein the one or more physiological sensors include one or more of the following: off-body physiological sensors and on-body physiological sensors.

10. The virtual test platform of claim 9, wherein the off-body physiological sensors include one or more of the following: audio sensors, cameras, thermal cameras, body markers, facial markers, pressure mats, and technology tracking sensors.

11. The virtual test platform of claim 9, wherein the on-body physiological sensors include one or more of the following: pressure sensors, galvanic skin response sensors, eye-tracking sensors, electroencephalography (EEG) sensors, electromyography (EMG) sensors, and functional near-infrared spectroscopy (FNIRS) sensors.

12. The virtual test platform of claim 7, wherein the one or more processors of the one or more controllers execute instructions to:
classify the physiological measurements of the individual into categories indicating the specific cognitive-affective state of the individual based on signal values generated by the one or more physiological sensors.

13. The virtual test platform of claim 7, wherein the one or more processors of the one or more controllers execute instructions to:
assign numerical values to signals generated by the one or more physiological sensors, wherein the numerical values represent the magnitude of the specific cognitive-affective state; and
index numerical values to a baseline value that represents a neutral cognitive-affective state, wherein the specific cognitive-affective state predicted by the one or more controllers is relative to the neutral specific cognitive-affective state.

14. The virtual test platform of claim 2, wherein the specific cognitive-affective state of the individual is predicted based on both the physiological measurements collected by the one or more physiological sensors and the one or more survey questions.

15. The virtual test platform of claim 14, wherein the one or more processors of the one or more controllers execute instructions to:
predict the specific cognitive-affective state of the individual based on a custom classification model is based on a recurrent neural network (RNN) having an internal memory.

16. The virtual test platform of claim 2, wherein the one or more recommendations include ranges of values for the one or more experimental parameters that result in the individual having a neutral cognitive-affective state while completing the assigned task within the workspace.

17. A virtual test platform for predicting a specific cognitive-affective state of an individual, the virtual test platform comprising:
a simulated environment generator that creates a computer-generated environment representing a workspace viewed by the individual, wherein the individual is required to complete an assigned task within the workspace simulated by the computer-generated environment;
an input device receiving user input generated by the individual, wherein the individual answers one or more survey questions either while performing or after performing the assigned task by the input device; and
one or more controllers in electronic communication with the simulated environment generator and the input device, wherein the one or more controllers include one or more processors that execute instructions to:
instruct the simulated environment generator to create the computer-generated environment representing the workspace;
predict the specific cognitive-affective state of the individual as the individual completes the assigned task created by the simulated environment generator based on user input received from the input device indicative of answers to the one or more survey questions from the individual;
instruct the simulated environment generator to modify one or more experimental parameters related to the assigned task within the workspace, wherein the individual is required to re-execute the assigned task within the workspace when the one or more experimental parameters that are related to the assigned task are modified;
instruct the simulated environment generator to create computer-generated perception information that asks the individual the one or more survey questions each time the one or more experimental parameters within the workspace are modified; and
formulate one or more recommendations to implement the workspace in a real-world environment based on the specific cognitive-affective state of the individual.

18. The virtual test platform of claim 17, wherein the one or more survey questions include a plurality of multiple-choice answers, and wherein each multiple-choice answer includes a numerical level representing a magnitude of the specific cognitive-affective state experienced by the individual.

19. The virtual test platform of claim 17, wherein the one or more survey questions include free-text responses.

20. A virtual test platform for predicting a specific cognitive-affective state of an individual, the virtual test platform comprising:
a simulated environment generator that creates a computer-generated environment representing a workspace viewed by the individual, wherein the individual is required to complete an assigned task within the workspace simulated by the computer-generated environment;
one or more physiological sensors that monitor physiological measurements of the individual; and
one or more controllers in electronic communication with the simulated environment generator and the one or more physiological sensors, wherein the one or more controllers include one or more processors that execute instructions to:
instruct the simulated environment generator to create the computer-generated environment representing the workspace;
predict the specific cognitive-affective state of the individual as the individual completes the assigned task created by the simulated environment generator based on the physiological measurements from the one or more physiological sensors;
instruct the simulated environment generator to modify one or more experimental parameters related to the assigned task within the workspace, wherein the individual is required to re-execute the assigned task within the workspace when the one or more experimental parameters that are related to the assigned task are modified;
continue to monitor the physiological measurements of the individual collected by the one or more physiological sensors as the one or more experimental parameters are modified; and
formulate one or more recommendations to implement the workspace in a real-world environment based on the specific cognitive-affective state of the individual.
